(19) United States
(12) Patent Application Publication
(10) Pub. No.: US 2004/0266772 A1
(43) Pub. Date: Dec. 30, 2004

(54) Polymorph salt of a pryridazinone derivative for the treatment of arrythmia

(76) Inventors: Jozsef Barkoczy, Budapest (HU); Peter Kotay Nagy, Vac (HU); Gyula Simig, Budapest (HU); Zsuzsa Szent-Kirallyi, Budapest (HU)

Correspondence Address: BIRCH STEWART KOLASCH & BIRCH, PO BOX 747, FALLS CHURCH, VA, 22040-0747, US

(21) Appl. No.: 10/484,621

(22) PCT Filed: Jul. 26, 2002
(86) PCT No.: PCT/HU02/00076

(30) Foreign Application Priority Data
Jul. 26, 2001 (HU) P0103064

(51) Int. Cl. A61K 31/50; C07D 237/02

(52) U.S. Cl. 514/247; 544/239

(57) ABSTRACT

The invention relates to the new crystalline form I 5-chloro-4-[3-[N-[2-(3,4-dimethoxy-phenyl)-ethyl]-N-methylamino]-propylamino]-3-[2H]-pyridazinone fumarate, a process for the preparation thereof, pharmaceutical compositions containing the same and the use of said new polymorph for the treatment of arrhythmia.

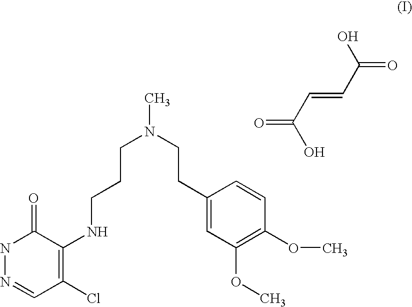

### FIELD OF THE INVENTION

This invention relates to a new polymorph salt, a process for the preparation thereof, pharmaceutical compositions containing the same and the use of said polymorph salt for the treatment of arrhythmia.

More particularly the present invention is concerned with the new crystalline form I 5-chloro-4-[3-[N-[2-(3,4-dimethoxy-phenyl)-ethyl]-N-methylamino]-propylamino]-3-[2H]-pyridazinone fumarate, a process for the preparation thereof, pharmaceutical compositions containing the same and the use of said new polymorph for the treatment of arrhythmia.

### TECHNICAL BACKGROUND

It is known that 5-chloro-4-[3-[N-[2-(3,4-dimethoxy-phenyl)-ethyl]-N-methylamino]-propylamino]-3-[2H]-pyridazinone fumarate is a useful antiarrhythmial agent which inhibits ventrical and auricular fibrillation.

5-chloro4-[3-[N-[2-(3,4-dimethoxy-phenyl)-ethyl]-N-methylamino]-propylamino]-3-[2H]-pyridazinone fumarate was described and characterized by physical constants (melting point) the first time in GB 2,262,526 and the corresponding HU 211,487. However, said patent specifications are completely silent in mentioning any crystal form or structure of the product. According to the example of GB 2,262,526 5-chloro-4-[3-[N-[2-(3,4-dimethoxy-phenyl)-ethyl]-N-methylamino]-propylamino]-3-[2 H]-pyridazinone fumarate is prepared as follows:

4,5-dichloro-3-[2H]-pyridazinone is reacted in dioxane with N-[2-(3,4-dimethoxy-phenyl)-ethyl]-N-methyl-1,3-propanediamine, whereupon the reaction mixture is refluxed for 10 hours and evaporated. The residual oil is subjected to chromatography on a silica column and eluted with a 9:1:0.5 mixture of ethyl acetate, methanol and concentrated ammonium hydroxide. Fumarate is then formed from the product thus obtained. The salt formation step is not described in the patent specification and it is only disclosed that the fumarate salts melts at 94-96° C.

In PCT patent application HU-98/00054 a new process for the preparation of 5-chloro-4-[3-[N-[2-(3,4-dimethoxy-phenyl)-ethyl]-N-methylamino]-propylamino]-3-[2H]-pyridazinone base is disclosed. Although it is mentioned in this document that the base can be converted into salts by methods known per se, the preparation of no salt is specifically described.

Thus the preparation of the fumarate salt has been specifically described in a concrete manner in none of the cited references. According to our experiments it has been found that when dissolving 5-chloro4-[3-[N-[2-(3,4-dimethoxy-phenyl)-ethyl]-N-methylamino]-propylamino]-3-[2H]-pyridazinone base in hot anhydrous ethanol, adding fumaric acid to the solution and evaporating the reaction mixture in vacuo a white foamy amorphous product melting at 97-99° C. is obtained. A comparison of the above melting point and the value disclosed in GB 2,262,526 indicates that according to the process disclosed in said reference actually the amorphous product was obtained.

It is known that the processing (e.g. tabletting) of amorphous active ingredients into pharmaceutical compositions is difficult and circumstantial because amorphous substances can be filtered and dried only in a difficult manner, the scaling up is encountered with difficulties and the stability and storability of the product is not satisfactory either.

### ESSENCE OF THE INVENTION

It is the object of the present invention to develop a new form of 5-chloro-4-[3-[N-[2-(3,4-dimethoxy-phenyl)-ethyl]-N-methylamino]-propylamino]-3-[2H]-pyridazinone fumarate which has favourable tabletting properties, can be easily filtered and dried, is stable for a long period of time and can be readily stored.

The above object is solved by the present invention.

The present invention is based on the recognition that 5-chloro-4-[3-[N-[2-(3,4-dimethoxy-phenyl)-ethyl]-N-methylamino]-propylamino]-3-[2H]-pyridazinone fumarate can be prepared in crystalline form which meets the above requirements.

### DETAILS OF THE INVENTION

The term “polymorph” used in the present patent specification relates to the new crystalline form I 5-chloro-4-[3-[N-[2-(3,4-dimethoxy-phenyl)-ethyl]-N-methylamino]-propylamino]-3-[2H]-pyridazinone fumarate.

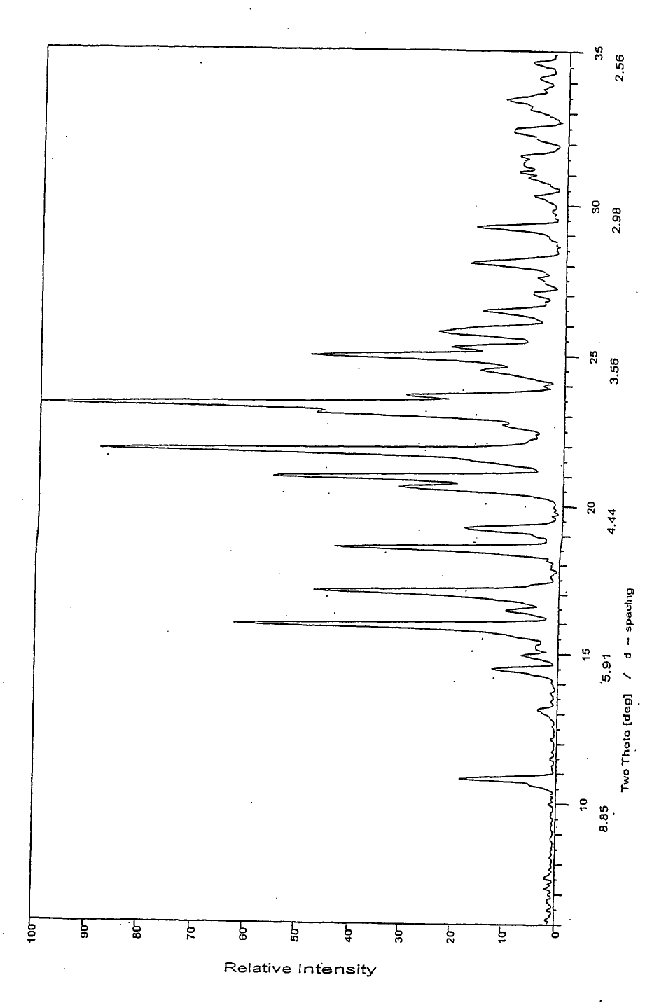

According to an aspect of the present invention there is provided crystalline form I 5-chloro-4-[3-[N-[2-(3,4-dimethoxy-phenyl)-ethyl]-N-methylamino]-propylamino]-3-[2H]-pyridazinone fumarate characterized by the X-ray powder diffraction pattern expressed in Table 1 and FIG. 1., measured using CuKα radiation:

TABLE 1
Position of diffraction lines and relative intensities (>10%)

| Peak | 2*th | d(hkl) | I(abs) | I(rel) |
|---|---|---|---|---|
| No. | [deg] | [Å] | [cts] | [%] |
| 1 | 10.84 | 8.1612 | 228 | 17.95 |
| 2 | 14.52 | 6.1006 | 153 | 12.05 |
| 3 | 15.95 | 5.5581 | 785 | 61.81 |
| 4 | 16.45 | 5.3880 | 120 | 9.45 |
| 5 | 17.10 | 5.1846 | 591 | 46.54 |
| 6 | 18.58 | 4.7752 | 544 | 42.83 |
| 7 | 19.26 | 4.6084 | 227 | 17.87 |
| 8 | 20.64 | 4.3043 | 392 | 30.87 |
| 9 | 20.97 | 4.2367 | 699 | 55.04 |
| 10 | 21.85 | 4.0685 | 1117 | 87.95 |
| 11 | 22.72 | 3.9135 | 141 | 11.10 |
| 12 | 23.08 | 3.8537 | 595 | 46.85 |
| 13 | 23.35 | 3.8093 | 1270 | 100.00 |
| 14 | 23.68 | 3.7574 | 377 | 29.69 |
| 15 | 24.52 | 3.6306 | 196 | 15.43 |
| 16 | 24.99 | 3.5638 | 611 | 48.11 |
| 17 | 25.28 | 3.5231 | 268 | 21.10 |
| 18 | 25.80 | 3.4536 | 300 | 23.62 |
| 19 | 26.49 | 3.3653 | 189 | 14.88 |
| 20 | 28.09 | 3.1762 | 222 | 17.48 |
| 21 | 29.27 | 3.0513 | 208 | 16.38 |
| 22 | 32.48 | 2.7567 | 121 | 9.53 |
| 23 | 33.44 | 2.6801 | 144 | 11.34 |

The powder diffraction pattern of the new crystalline form I 5-chloro-4-[3-[N-[2-(3,4-dimethoxy-phenyl)-ethyl]-N-methylamino]-propylamino]-3-[2H]-pyridazinone fumarate of the present invention is determined under the following conditions:

Equipment: PHILIPS—XPERT PW 3710 powder diffractometer

Radiation: CuKα (λ: 1.54190L)

Monochromator: graphite

Exciting voltage: 40 kV

Anode current: 30 Ma

Standard reference substance: SRM 675

Mica Powder (synthetic fluorographite), Ser. No.: 981307.

The measurement is continuous: E120 scan: 6.00°-35.00° 2Θ

step size: 0.04°

Sample: surface plain, width 0.5 mm, in quartz sample holder, measured and stored at room temperature.

According to another aspect of the present invention there is provided a process for the preparation of crystalline form I 5-chloro-4-[3-[N-[2-(3,4-dimethoxy-phenyl)-ethyl]-N-methylamino]-propylamino]-3-[2H]-pyridazinone fumarate which comprises

a) dissolving 5-chloro-4-[3-(N-[2-(3,4-dimethoxy-phenyl)-ethyl]-N-methylamino]-propylamino]-3-[2H]-pyridazinone and fumaric acid in an inert solvent under heating, slowly cooling the solution to room temperature, and thereafter isolating the precipitated crystalline polymorph; or

b) dissolving amorphous 5-chloro4-[3-[N-[2-(3,4-dimethoxy-phenyl)-ethyl]-N-methylamino]-propylamino]-3-[2H3-pyridazinone fumarate in an inert solvent under heating, slowly cooling the solution to room temperature, and thereafter isolating the precipitated crystalline polymorph.

According to process a) 5-chloro-4-[3-[N-[2-(3,4-dimethoxy-phenyl)-ethy)]-N-methylamino]-propylamino]-3-[2H]-pyridazinone base and fumaric acid are dissolved in an inert solvent under heating, preferably under heating at reflux temperature.

According to process a) a protic, dipolar aprotic or apolar solvent is used as inert solvent. As protic solvent preferably a lower alkanol (advantageously methanol or ethanol) or water or a mixture thereof can be used. From the dipolar aprotic solvents which can be used in the process acetone, ethyl acetate, acetonitrile, dimethyl formamide, dimethyl sulfoxide or hexamethyl phosphoric triamide can be mentioned. As apolar solvent a halogenated hydrocarbon (preferably dichloro methane, dichloro ethane or chloroform) can be used. It is preferred to use methanol, ethanol, water, acetonitrile, ethyl acetate, dichloro methane, a mixture of ethanol and water, a mixture of ethanol and acetonitrile, a mixture of methanol and ethyl acetate or a mixture of ethanol and dichloro methane as inert solvent.

In the salt formation step 5-chloro-4-[3-[N-[2-(3,4-dimethoxy-phenyl)-ethyl]-N-methylamino]-propylamino]-3-[2H]-pyridazinone base and fumaric acid may preferably be used in equimolar amount.

The fumarate solution thus obtained is then slowly cooled to room temperature. The duration of the cooling step is 1.5-24 hours, preferably 2-5 hours. On cooling crystalline form I 5-chloro-4-[3-[N-[2-(3,4-dimethoxy-phenyl)-ethyl]-N-methylamino]-propylamino]-3-[2H]-pyridazinone fumarate is precipitated, which is then isolated, preferably by means of filtration or centrifuging.

According to process b) amorphous 5-chloro-4-[3-[N-[2-(3,4-dimethoxy-phenyl)-ethyl]-N-methylamino]-propylamino]-3-[2H]-pyridazinone fumarate is dissolved in an inert solvent under heating, the solution is then slowly cooled to room temperature and the precipitated crystalline polymorph is isolated. The reaction conditions (inert solvent, speed of cooling) are the same as those disclosed by process a).

According to a further aspect of the present invention there is provided a pharmaceutical composition comprising crystalline form I 5-chloro4-[3-[N-[2-(3,4-dimethoxy-phenyl)-ethyl]-N-methylamino]-propylamino]-3-[2H)-pyridazinone fumarate as active ingredient in admixture with inert, solid or liquid pharmaceutical carriers and/or auxiliary agent.

The pharmaceutical compositions according to the present invention can be administered orally, parenterally, by inhalation or transdermally. The pharmaceutical compositions can also be administered by injection (preferably intravenously, intramuscularly, intracutantly, subcutantly, intraduodenally or intraperitoneally) or by inhalation (e.g. intranasally) or transdermally.

The pharmaceutical compositions according to the present invention can be preferably prepared in the form of tablets, capsules, piluies, powders, lozenges, sachets, suppositories, dispersible granules, solutions, emulsions, suspensions or spray.

The pharmaceutical compositions of the present invention can be prepared by admixing crystalline form I 5-chloro-4-[3-[N-[2-(3,4-dimethoxy-phenyl)-ethyl]-N-methylamino]-propylamino]-3-[2H]-pyridazinone fumarate with pharmaceutically acceptable solid or liquid carriers and/or auxiliary agents and bringing the mixture to galenic form.

The pharmaceutical compositions of the present invention can be prepared by conventional methods of pharmaceutical industry.

The pharmaceutical compositions according to the present invention contain usual pharmaceutical carriers and/or auxiliary agent. As carrier e.g. magnesium carbonate, magnesium stearate, talc, saccharose, lactose, pectine, dextrine, starch, gelatine, tragacanth, methyl cellulose, sodium carboxymethyl cellulose, low melting wax, cocoa buffer etc. can be used. In case of capsules the carrier is generally the wall of the capsule so that no additional carrier is needed. As oral-administration form the lozenge and sachet can be also mentioned. Tablets, powders, capsules, pilules, sachets and lozenges are solid forms particularly suitable for oral administration.

Suppositories may contain low melting waxes (e.g. mixtures of fatty acid triglycerides or cocoa butter) as carrier. Suppositories can be prepared by melting the wax, homogeneously distributing the active ingredient in the melt, pouring the melt homogenous mixture into mould forms of suitable size and form, and allowing the mixture to solidify under cooling.

Tablets can be prepared by admixing the active ingredient with suitable carriers in the appropriate ratio and pressing the mixture into tablets of suitable form and size.

Powders are prepared by admixing the finely powdered active ingredient with finely powdered carriers.

As liquid pharmaceutical compositions optionally sustained release solutions, suspensions and emulsions can be mentioned. Aqueous solutions and aqueous propylene glycol solutions are advantageous. Liquid pharmaceutical compositions suitable for parenteral administration can be preferably prepared in the form of aqueous polyethylene glycol solutions.

Aqueous solutions suitable for oral administration can be prepared by dissolving the active ingredient in water and adding suitable colouring, aromatizing, stabilizing agents and thickeners.

Aqueous suspensions suitable for oral administration can be prepared by suspending the active ingredient in water in the presence of a viscous substance (e.g. natural or artificial gums, resins, methyl cellulose, sodium carboxymethyl cellulose or other known suspending agents).

Another type of solid pharmaceutical compositions can be converted into liquid compositions immediately before use and orally administered into the organism in liquid form. Solutions, suspensions and emulsions can be mentioned as such liquid forms of administration which can contain in addition to the active ingredient colouring agents, aromatising agents, preservatives, buffers, artificial or natural sweeteners, dispersing agents, thickeners etc.

The pharmaceutical compositions of the present invention are preferably prepared in dosage unit form. Such dosage units contain the desired amount of the active ingredient. The dosage units can be put on the market in packages which contain discrete amounts of the compositions (e.g. packed tablets, capsules, or powders in vials or ampouls). The term “dosage” unit relates to the capsules, tablets, lozenges, sachets per se and also to the packaging which contains the suitable number of dosage units.

The pharmaceutical compositions of the present invention can optionally contain one or more further pharmaceutical active ingredients compatible with the crystalline form I 5-chloro-4-[3-[N-[2-(3,4-dimethoxy-phenyl)-ethyl]-N-methylamino]-propylamino]-3-[2H]-pyridazinone fumarate.

The daily dosage of crystalline form I 5-chloro-4-[3-[N-[2-(3,4-dimethoxy-phenyl)-ethyl]-N-methylamino3-propylamino]-3-[2H]-pyridazinone fumarate of the present invention depends on the circumstances of the given case (e.g. the seriousness of the illness to be treated, the condition and body weight of the patient etc.) and is determined by the physician.

According to a still further aspect of the present invention there is provided crystalline form I 5-chloro-4-[3-[N-[2-(3,4-dimethoxy-phenyl)-ethyl]-N-methylamino]-propylamino]-3-[2H]-pyridazinone fumarate, for use as pharmaceutical active ingredient.

According to a still further aspect of the present invention there is provided the use of crystalline form I 5-chloro-4-[3-[N-[2-(3,4-dimethoxy-phenyl)-ethyl]-N-methylamino]-propylamino]-3-[2H]-pyridazinone fumarate for the preparation of antiarrhythmial pharmaceutical compositions.

According to a still further aspect of the present invention there is provided the use of crystalline form I 5-chloro-4-[3-[N-[2-(3,4-dimethoxy-phenyl)-ethyl]-N-methylamino]-propylamino]-3-[2H]-pyridazinone fumarate for the treatment of arrhythmia.

According to a still further aspect of the present invention there is provided method for the treatment of arrhythmia which comprises administering to the patient in need of such treatment a pharmaceutically active amount of crystalline form I 5-chloro4-[3-[N-(2-(3,4-dimethoxy-phenyl)-ethyl]-N-methylamino]-propylamino]-3-[2H]-pyridazinone fumarate.

Further details of the present invention are to be found in the following Examples, without limiting the scope of protection to said Examples.

### EXAMPLE 1

### Preparation of crystalline form I 5-chloro-4-[3-[N-[2-(3,4-dimethoxy-phenyl)-ethyl]-N-methylamino]-propylamino]-3-[2H]-pyridazinone fumarate

To 3.8 g (0.01 mole) of 5-chloro-4-[3-[N-[2-(3,4-dimethoxy-phenyl)-ethyl]-N-methylamino]-propylamino]-3-[2H]-pyridazinone base at first a mixture of 30 ml of methanol and 60 ml of ethyl acetate, and thereafter 1.16 g (0.01 mole) of anhydrous fumaric acid are added. The reaction mixture is heated to boiling for 10 minutes under stirring, then cooled to room temperature within 2 hours under stirring, while the solution is seeded with crystalline form I polymorph. The suspension thus obtained is stirred at room temperature for 3 hours. The precipitated crystals are filtered and dried at 40° C. in vacuo. Thus 4.22 g of crystalline form I 5-chloro-4-[3-[N-[2-(3,4-dimethoxy-phenyl)-ethyl]-N-methylamino]-propylamino]-3-[2H]-pyridazinone fumarate are obtained in the form of a white product. Yield 85 %, mp.: 177-178° C.

### EXAMPLE 2

### Preparation of crystalline form I 5-chloro-4-[3-[N-[2-(3,4-dimethoxy-phenyl)-ethyl]-N-methylamino]-propylamino]-3-[2H]-pyridazinone fumarate

To 3.8 g (0.01 mole) of 5-chloro-4-[3-[N-[2-(3,4-dimethoxy-phenyl)-ethyl]-N-methylamino]-propylamino]-3-[2H]-pyridazinone base at first a mixture of 50 ml of methanol and 10 ml of water, and thereafter 1.16 g (0.01 mole) of anhydrous fumaric acid are added. The reaction mixture is heated to boiling for 10 minutes under stirring, then cooled to room temperature within 3 hours under stirring, while the solution is seeded with crystalline form I polymorph. The suspension thus obtained is stirred at room temperature for 4 hours. The precipitated crystals are filtered and dried at 40° C. in vacuo. Thus 4.42 g of white crystalline form I 5-chloro4-[3-[N-[2-(3,4-dimethoxy-phenyl)-ethyl]-N-methylamino]-propylamino]-3-[2H]-pyridazinone fumarate are obtained in the form of a white product. Yield 89 %, mp.: 177-178° C.

### EXAMPLE 3

### Preparation of crystalline form I 5-chloro-4-[3-]N-[2-(3,4-dimethoxy-phenyl)-ethyl]-N-methylamino]-propylamino]-3-[2H]-pyridazinone fumarate

To 3.8 g (0.01 mole) of 5-chloro-4-[3-[N-[2-(3,4-dimethoxy-phenyl)-ethyl]-N-methylamino]-propylamino]-3-[2H]-pyridazinone base at first a mixture of 5 ml of ethanol and 30 ml of water, and thereafter 1.16 g (0.01 mole) of anhydrous fumaric acid are added. The reaction mixture is heated to boiling for 10 minutes under stirring, then cooled to room temperature within an hour under stirring, while the solution is seeded with crystalline form I polymorph. The suspension thus obtained is stirred at room temperature for 8 hours. The precipitated crystals are filtered and dried at 40° C. in vacuo. Thus 3.98 g of white crystalline form I 5-chloro-4-[3-[N-[2-(3,4-dimethoxy-phenyl)-ethyl]-N-methylamino]-propylamino]-3-[2H]-pyridazinone fumarate are obtained. Yield 80 %, mp.: 177-178° C.

### EXAMPLE 4

### Preparation of crystalline form I 5-chloro-4-[3-[N-[2-(3,4-dimethoxy-phenyl)-ethyl]-N-methylamino]-propylamino]-3-[2H]-pyridazinone fumarate

To 3.8 g (0.01 mole) of 5-chloro-4-[3-[N-[2-(3,4-dimethoxy-phenyl)-ethyl]-N-methylamino]-propylamino]-3-[2H]-pyridazinone base at first 60 ml of water and thereafter 1.16 g (0.01 mole) of anhydrous fumaric acid are added. The reaction mixture is heated to boiling for 10 minutes under stirring, then cooled to room temperature under stirring within 2 hours, while the solution is seeded with crystalline form I 5-chloro-4-[3-[N-[2-(3,4-dimethoxy-phenyl)-ethyl]-N-methylamino]-propylamino]-3-[2H]-pyridazinone fumarate. The suspension is stirred at room temperature for 5 hours. The precipitated crystals are filtered and dried at 40° C. in vacuo. Thus 3.93 g of white crystalline form I 5-chloro-4-[3-[N-[2-(3,4-dimethoxy-phenyl)-ethyl]-N-methylamino]-propylamino]-3-[2H]-pyridazinone fumarate are obtained. Yield 79 %, mp.: 177-178° C.

### EXAMPLE 5

### Preparation of crystalline form I 5-chloro-4-[3-[N-[2-(3,4-dimethoxy-phenyl)-ethyl]-N-methylamino]-propylamino]-3-[2H]-pyridazinone fumarate

To 3.8 g (0.01 mole) 5-chloro-4-[3-[N-[2-(3,4-dimethoxy-phenyl)-ethyl]-N-methylamino]-propylamino]-3-[2H]-pyridazinone base at first a mixture of 250 ml of acetonitrile and 200 ml of ethanol, and thereafter 1.16 g (0.01 mole) of anhydrous fumaric acid are added. The reaction mixture is heated to boiling for 10 minutes under stirring, then cooled to room temperature within an hour under stirring, while the solution is seeded with crystalline form I 5-chloro-4-[3-[N-[2-(3,4-dimethoxy-phenyl)-ethyl]-N-methylamino]-propylamino]-3-[2H]-pyridazinone fumarate. The suspension thus obtained is stirred at room temperature for 5 hours. The precipitated crystals are filtered and dried at 40° C. in vacuo. Thus 4.57 g of white crystalline form I 5-chloro-4-[3-[N-(2-(3,4-dimethoxy-phenyl)-ethyl]-N-methylamino]-propylamino]-3-[2H]-pyridazinone fumarate are obtained. Yield 92 %, mp.: 177-178° C.

### EXAMPLE 6

### Preparation of crystalline form I 5-chloro4-3-[N-[2-(3,4-dimethoxy-phenyl)-ethyl]-N-methylamino]-propylamino]-3-[2H]-pyridazinone fumarate

To 3.8 g (0.01 mole) of 5-chloro-4-[3-[N-[2-(3,4-dimethoxy-phenyl)-ethyl]-N-methylamino]-propylamino]-3-[2H]-pyridazinone base at first a mixture of 250 ml of dichloro methane and 350 ml of ethanol, and thereafter 1.16 g (0.01 mole) of anhydrous fumaric acid are added. The reaction mixture is heated to boiling for 10 minutes under stirring, then cooled to room temperature within an hour under stirring, while the solution is seeded with crystalline form I 5-chloro-4-[3-[N-[2-(3,4-dimethoxy-phenyl)-ethyl]-N-methylamino)-propylamino]-3-[2H]-pyridazinone fumarate. The suspension thus obtained is stirred at room temperature for 5 hours. The precipitated crystals are filtered and dried at 40° C. in vacuo. Thus 4.08 g of white crystalline form I 5-chloro-4-[3-[N-[2-(3,4-dimethoxy-phenyl)-ethyl]-N-methylamino]-propylamino]-3-[2H]-pyridazinone fumarate are obtained. Yield 82 %, mp.: 177-178° C.

### EXAMPLE 7

### Preparation of crystalline form I 5-chloro-4-[3-[N-[2-(3,4-dimethoxy-phenyl)-ethyl]-N-methylamino]-propylamino]-3-[2H]-pyridazinone fumarate

3.8 g (0.01 mole) of 5-chloro-4-[3-[N-[2-(3,4-dimethoxy-phenyl)-ethyl]-N-methylamino]-propylamino]-3-[2H]-pyridazinone base are dissolved in 53 ml of hot anhydrous ethanol, whereupon at 77° C. 1.16 g (0.01 mole) of anhydrous fumaric acid are added. The reaction mixture is heated to boiling for 10 minutes under stirring, then cooled to room temperature within 2 hours under stirring. The precipitated crystals are filtered, washed with some ethanol and dried at 40° C. in vacuo. Thus 4.86 g of white crystalline form I 5-chloro-4-[3-[N-[2-(3,4-dimethoxy-phenyl)-ethyl]-N-methylamino]-propylamino]-3-[2H]-pyridazinone fumarate are obtained. Yield 97.8 %, mp.: 177-178° C.

### EXAMPLE 8

### Preparation of crystalline form I 5-chloro-4-[3-[N-[2-(3,4-dimethoxy-phenyl)-ethyl]-N-methylamino]-propylamino]-3-[2H]-pyridazinone fumarate

4.97 g (0.01 mole) of amorphous 5-chloro-4-[3-[N-[2-(3,4-dimethoxy-phenyl)-ethyl]-N-methylamino]-propylamino]-3-[2H]-pyridazinone fumarate are dissolved in a mixture of 30 ml of methanol and 60 ml of ethyl acetate under heating at reflux temperature. The mixture is cooled to room temperature within 2 hours under stirring. The solution is seeded with crystalline form I polymorph. The precipitated crystals are filtered and dried at 40° C. in vacuo. Thus 3.53 g of white crystalline form I 5-chloro4-[3-[N-[2-(3,4-dimethoxy-phenyl)-ethyl]-N-methylamino]-propylamino]-3-[2H]-pyridazinone fumarate are obtained. Yield 71 %, mp.: 177-178° C.

### EXAMPLE 9

### Preparation of crystalline form I 5-chloro-4-[3-[N-[2-(3,4-dimethoxy-phenyl)-ethyl]-N-methylamino]-propylamino]-3-[2H]-pyridazinone fumarate

4.97 g (0.01 mole) of amorphous 5-chloro-4-[3-[N-[2-(3,4-dimethoxy-phenyl)-ethyl]-N-methylamino]-propylamino]-3-[2H]-pyridazinone fumarate are dissolved in a mixture of 50 ml of ethanol and 10 ml of water under heating at reflux temperature. The reaction mixture is cooled to room temperature within an hour under stirring. The solution is seeded with crystalline form I polymorph. The precipitated crystals are filtered and dried at 40° C. in vacuo. Thus 4.42 g of white crystalline form I 5-chloro-4-[3-[N-[2-(3,4-dimethoxy-phenyl)-ethyl]-N-methylamino]-propylamino]-3-[2H]-pyridazinone fumarate are obtained. Yield 89 %, mp.: 177-178° C.

### EXAMPLE 10

### Preparation of crystalline form I 5-chloro-4-[3-[N-[2-(3,4-dimethoxy-phenyl)-ethyl]-N-methylamino]-propylamino]-3-[2H]-pyridazinone fumarate

4.97 g (0.01 mole) of amorphous 5-chloro-4-[3-[N-[2-(3,4-dimethoxy-phenyl)-ethyl]-N-methylamino]-propylamino]-3-[2H]-pyridazinone fumarate are dissolved in a mixture of 5 ml of ethanol and 30 ml of water under heating at reflux temperature. The mixture is cooled to room temperature within an hour under stirring. The solution is seeded with crystalline form I polymorph. The precipitated crystals are filtered and dried at 40° C. in vacuo. Thus 3.98 g of white crystalline form I 5-chloro-4-[3-[N-[2-(3,4-dimethoxy-phenyl)-ethyl]-N-methylamino]-propylamino]-3-[2H]-pyridazinone fumarate are obtained. Yield 80 %, mp.: 177-178° C.

### EXAMPLE 11

### Preparation of crystalline form I 5-chloro4-[3-[N-[2-(3,4-dimethoxy-phenyl)-ethyl]-N-methylamino]-propylamino]-3-[2H]-pyridazinone fumarate

4.97 g (0.01 mole) of amorphous 5-chloro-4-[3-[N-[2-(3,4-dimethoxy-phenyl)-ethyl]-N-methylamino]-propylamino]-3-[2H]-pyridazinone fumarate are dissolved in 60 ml of water under heating at reflux temperature. The mixture is cooled to room temperature within 2 hours under stirring. The solution is seeded with crystalline form I polymorph. The precipitated crystals are filtered and dried at 40° C. in vacuo. Thus 3.93 g of white crystalline form I 5-chloro-4-[3-[N-[2-(3,4-dimethoxy-phenyl)-ehtyl]-N-methylamino]-propylamino]-3-[2H]-pyridazinone fumarate are obtained. Yield 79%, mp.: 177-178° C.

### EXAMPLE 12

### Preparation of crystalline form I 5-chloro-4-3-[N-[2-(3,4-dimethoxy-phenyl)-ethyl]-N-methylamino]-propylamino]-3-[2H]-pyridazinone fumarate

4.97 g (0.01 mole) of amorphous 5-chloro-4-[3-[N-[2-(3,4-dimethoxy-phenyl)-ethyl]-N-methylamino]-propylamino]-3-[2H]-pyridazinone fumarate are dissolved in a mixture of 250 ml of acetonitrile and 200 ml of ethanol under heating at reflux temperature. The mixture is cooled to room temperature within an hour under stirring. The solution is seeded with crystalline form I polymorph. The precipitated crystals are filtered and dried at 40° C. in vacuo. Thus 4.57 g of white crystalline form I 5-chloro-4-[3-[N-[2-(3,4-dimethoxy-phenyl)-ethyl]-N-methylamino]-propylamino]-3-[2H]-pyridazinone fumarate are obtained. Yield 92%, mp.: 177-178° C.

### EXAMPLE 13

### Preparation of crystalline form I 5-chloro-4-[3-[N-[2-(3,4-dimethoxy-phenyl)-ethyl]-N-methylamino]-propylamino]-3-[2H]-pyridazinone fumarate

4.97 g (0.01 mole) of amorphous 5-chloro-4-[3-[N-[2-(3,4-dimethoxy-phenyl)-ethyl]-N-methylamino]-propylamino]-3-[2H]-pyridazinone fumarate are dissolved in a mixture of 250 ml of dichloro methane and 350 ml of ethanol under heating at reflux temperature. The mixture is cooled to room temperature within an hour under stirring. The solution is seeded with crystalline form I polymorph. The precipitated crystals are filtered at 40° C. and dried in vacuo. Thus 4.08 g of white crystalline form I 5-chloro-4-[3-[N-[2-(3,4-dimethoxy-phenyl)-ethyl]-N-methylamino]-propylamino]-3-[2H]-pyridazinone fumarate are obtained. Yield 82%, mp.: 177-178° C.

What we claim is,

1. Crystalline form I 5-chloro-4-[3-[N-[2-(3,4-dimethoxy-phenyl)-ethyl]-N-methylamino]-propylamino]-3-[2H]-idazinone fumarate characterized by the x-ray powder diffraction pattern expressed in Table 1 and FIG. 1, measured using CuKα radiation:
TABLE 1
Position of diffraction lines and relative intensities (>10%)

| Peak | 2*th | d(hkl) | I(abs) | I(rel) |
|---|---|---|---|---|
| No. | [deg] | [Å] | [cts] | [%] |
| 1 | 10.84 | 8.1612 | 228 | 17.95 |
| 2 | 14.52 | 6.1006 | 153 | 12.05 |
| 3 | 15.95 | 5.5581 | 785 | 61.81 |
| 4 | 16.45 | 5.3880 | 120 | 9.45 |
| 5 | 17.10 | 5.1846 | 591 | 46.54 |
| 6 | 18.58 | 4.7752 | 544 | 42.83 |
| 7 | 19.26 | 4.6084 | 227 | 17.87 |
| 8 | 20.64 | 4.3043 | 392 | 30.87 |
| 9 | 20.97 | 4.2367 | 699 | 55.04 |
| 10 | 21.85 | 4.0685 | 1117 | 87.95 |
| 11 | 22.72 | 3.9135 | 141 | 11.10 |
| 12 | 23.08 | 3.8537 | 595 | 46.85 |
| 13 | 23.35 | 3.8093 | 1270 | 100.00 |
| 14 | 23.68 | 3.7574 | 377 | 29.69 |
| 15 | 24.52 | 3.6306 | 196 | 15.43 |
| 16 | 24.99 | 3.5638 | 611 | 48.11 |
| 17 | 25.28 | 3.5231 | 268 | 21.10 |
| 18 | 25.80 | 3.4536 | 300 | 23.62 |
| 19 | 26.49 | 3.3653 | 189 | 14.88 |
| 20 | 28.09 | 3.1762 | 222 | 17.48 |
| 21 | 29.27 | 3.0513 | 208 | 16.38 |
| 22 | 32.48 | 2.7567 | 121 | 9.53 |
| 23 | 33.44 | 2.6801 | 144 | 11.34 |

2. Process for the preparation of crystalline form I 5-chloro-4-[3-[N-[2-(3,4-dimethoxy-phenyl)-ethyl]-N-methylamino]-propylamino]-3-[2H]-pyridazinone fumarate according to claim 1 which comprises
a) dissolving 5-chloro-4-[3-[N-[2-(3,4-dimethoxy-phenyl)-ethyl]-N-methylamino]-propylamino]-3-[2H]-pyridazinone and fumaric acid in an inert solvent under heating, slowly cooling the solution to room temperature, and thereafter isolating the precipitated crystalline polymorph; or
b) dissolving amorphous 5-chloro4-[3-[N-[2-(3,4-dimethoxy-phenyl)-ethyl]-N-methylamino]-propylamino]-3-[2H]-pyridazinone fumarate in an inert solvent under heating, slowly cooling the solution to room temperature, and thereafter isolating the precipitated crystalline polymorph.

3. Process according to claim 2 which comprises using a protic, dipolar aprotic, or apolar solvent or a mixture thereof as inert solvent.

4. Process according to claim 3 which comprises using a lower alkanol or water or a mixture thereof as protic solvent.

5. Process according to claim 4 which comprises using methanol or ethanol as lower alkanol.

6. Process according to claim 2 which comprises using acetone, ethyl acetate, acetonitrile, dimethyl formamide, dimethyl sulfoxide or hexamethyl phosphoric triamide as dipolar aprotic solvent.

7. Process according to claim 2 which comprises using a halogenated hydrocarbon as apolar solvent.

8. Process according to claim 7 which comprises using dichloro methane, dichloro ethane or chloroform as halogenated hydrocarbon.

9. Process according to any of claims 2-8 which comprises using methanol, ethanol, water, acetonitrile, ethyl acetate, dichloro methane, a mixture of ethanol and water, a mixture of ethanol and acetonitrile, a mixture of methanol and ethyl acetate or a mixture of ethanol and dichloro methane as inert solvent.

10. Pharmaceutical composition comprising crystalline form I 5-chloro-4-[3-[N-[2-(3,4-dimethoxy-phenyl)-ethyl]-N-methylamino]-propylamino]-3-[2H]-pyridazinone fumarate as active ingredient in admixture with inert, solid or liquid pharmaceutical carriers and/or auxiliary agent.

11. Pharmaceutical composition in a form to be administered orally, parenterally, transdermally or by inhalation.

12. Pharmaceutical composition according to claim 11 in the form of tablets, capsules, pilules, powders, lozenges, sachets, suppositories, dispersible granules, solutions, emulsions, suspensions or spray.

13. Process for the preparation of pharmaceutical compositions according to claim 10 which comprises admixing crystalline form I 5-chloro-4-[3-[N-[2-(3,4-dimethoxy-phenyl)-ethyl]-N-methylamino]-propylamino]-3-[2H]-pyridazinone fumarate with pharmaceutically acceptable solid or liquid carriers and/or auxiliary agents and bringing the mixture to galenic form.

14. Crystalline form I 5-chloro-4-[3-[N-[2-(3,4-dimethoxy-phenyl)-ethyl]-N-methylamino]-propylamino]-3-[2H]-pyridazinone fumarate, for use as pharmaceutical active ingredient.

15. Use of crystalline form I 5-chloro-4-[3-[N-[2-(3,4-dimethoxy-phenyl)-ethyl]-N-methylamino]-propylamino]-3-[2H]-pyridazinone fumarate for the preparation of antiarrhythmial pharmaceutical compositions.

16. Use of crystalline form I 5-chloro-4-[3-[N-[2-(3,4-dimethoxy-phenyl)-ethyl]-N-methylamino]-propylamino]-3-[2H]-pyridazinone fumarate for the treatment of arrhythmia.

17. Method for the treatment of arrhythmia which comprises administering to the patient in need of such treatment a pharmaceutically active amount of crystalline form I 5-chloro-4-[3-[N-[2-(3,4-dimethoxy-phenyl)-ethyl]-N-methylamino]-propylamino]-3-[2H]-pyridazinone fumarate.
